(19) United States
(12) Patent Application Publication
(10) Pub. No.: US 2015/0197462 A1
(43) Pub. Date: Jul. 16, 2015

(54) PROCESS FOR PREPARING HYDROCARBONS FROM CARBON DIOXIDE AND HYDROGEN, AND A CATALYST USEFUL IN THE PROCESS

(71) Applicant: Edmund Wagner, Wiesbaden (DE)

(72) Inventors: Edmund Wagner, Wiesbaden (DE); Manfred Baerns, Berlin (DE); Uwe Rodemerck, Rostock (DE); Quido Smejkal, Berlin (DE); Axel Barkschat, Rostock (DE); Martin Holena, Rostock (DE); David Linke, Rostock (DE); Martina Marschall, Rostock (DE)

(21) Appl. No.: 14/414,601

(22) PCT Filed: Jul. 12, 2013
(86) PCT No.: PCT/EP2013/064806

§ 371 Date: Jan. 13, 2015

(30) Foreign Application Priority Data
Jul. 13, 2012 (EP) 12176417.9

(51) Int. Cl. C07C 1/12; B01J 23/80; B01J 23/72; B01J 23/76

(52) U.S. Cl. CPC C07C 1/12; B01J 23/76; B01J 23/80; B01J 23/72

(57) ABSTRACT

The invention relates to a process for producing hydrocarbons from carbon dioxide and hydrogen, wherein carbon dioxide is converted with hydrogen in the presence of a special catalyst. By means of the process according to the invention C₅-C₁₅ hydrocarbons which are of interest as fuels (benzine, diesel, kerosene) can be formed with very high selectivity. Furthermore, in the process C₂-C₄ hydrocarbons, which can be used as valuable starting materials in the chemical industry, are produced with good selectivity. In the process a high conversion of CO₂ is already achieved without any recirculation of excess carbon dioxide. According to another aspect, the invention relates to the catalysts as such.

### FIELD OF THE INVENTION

The present invention relates to a process for producing hydrocarbons from carbon dioxide and hydrogen, wherein carbon dioxide is converted with hydrogen in the presence of a special catalyst. By means of the process according to the invention C₅-C₁₅ hydrocarbons which are of interest as fuels (benzine, diesel, kerosene) can be formed with very high selectivity. Furthermore, in the process C₂-C₄ hydrocarbons, which can be used as valuable starting materials in the chemical industry, are produced with good selectivity. In the process a high conversion of CO₂ is already achieved without any recirculation of excess carbon dioxide. According to another aspect, the invention relates to the catalysts as such.

### PRIOR ART

The globally decreasing supplies of fossil carbon carriers and the increasing enrichment of the atmosphere with carbon dioxide (CO₂) and the resulting greenhouse effect (global warming) necessitate rethinking in the use of fossil carbon as an energy carrier and starting material for the production of fuels. There is currently a changeover in fuel production from crude oil- to natural gas-based processes. For this purpose the production of synthesis gas from methane and the catalyzed Fischer Tropsch synthesis (FT) are combined.

### Fischer Tropsch Synthesis:

CO+2H₂→[—CH₂—]+H₂O (1)

Shorter chain (benzine, diesel) and longer-chain hydrocarbons (waxes) can be obtained as products. Benzine contains hydrocarbons with 5 to 12 carbon atoms, whereas diesel contains longer chain hydrocarbons, namely with at least approx. 9 carbon atoms. The FT synthesis itself contributes to the CO₂ emission because CO₂ is produced as a by-product in a number of equilibrium reactions e.g. from carbon monoxide and water vapor in the water-gas shift:

CO+H₂O⇄CO₂+H₂ (2)

Therefore, processes for the production of fuels are sought which are CO₂ neutral. CO₂, for example, is suitable here as the carbon source because CO₂ is consumed during production and only the previously bonded amount of CO₂ is released when the fuel obtained is burnt.

One approach to this is to convert CO₂ with hydrogen into CO and water in the reverse water-gas shift (RWGS):

CO₂+H₂⇄CO+H₂O (3)

and then to convert the CO obtained into hydrocarbons in the Fischer-Tropsch reaction by hydrogenation. The overall reaction can then be expressed as:

CO₂+3H₂→[—CH₂—]+2H₂O (4)

The RWGS or its inverse reaction is an equilibrium reaction which is limited by the temperature and the ratio of the partial pressures. An H₂:CO₂ ratio of 3 corresponds to the stoichiometric ratio of the overall reaction, see equation (4). The CO₂ conversion and the CO yield increase with the temperature; only at approx. 500° C. is 50% of the CO₂ converted into CO, and temperatures of well over 1000° C. are required for total conversion. Over the temperature range of 220-300° C. the portion of CO₂ converted into CO is 12-22%.

In addition to fuel production, the greatest consumer of fossil carbon carriers in the chemical industry is the production of polymer. Short-chain olefins (ethylene, propylene, butene) are predominantly used here as starting materials from which polymers such as polyethylene, polypropylene, polyacrylates and polyurethanes are produced directly or after modification. The aforementioned olefins are currently mainly produced from fossil raw materials in refineries by steam cracking Other processes, such as the catalytic dehydrogenation of alkanes or the conversion of olefins into other olefins by metathesis only play a subordinate role and are also based on fossil raw materials. In order to become independent of fossil raw materials it is desirable to produce hydrocarbons for the production of fuels and to produce short-chain olefins from the raw material CO₂.

Another approach for reducing the enrichment of CO₂ in the atmosphere and the resulting greenhouse effect (global warming) is to store CO₂ in underground caverns. However, the CO₂ may react with the stone here and endanger the stability of the caverns.

It is desirable to convert the CO₂ into liquid hydrocarbons (more than 5 carbon atoms) which are then stored in the caverns and, if required, can be used as a fuel or raw material resource for the chemical industry.

In an earlier study by G. D. Weatherbee et al. (J. Catal. 87 (1984) 352) the hydrogenation of CO₂ by cobalt, iron and ruthenium was described, the latter being supported on silica. In D. Li et al., Appl. Catal. A 180 (1999) 227 the production of methane from CO₂ and H₂ in the presence of ruthenium on a TiO₂ carrier was investigated.

Various publications by T. Riedel et al. and H. Schulz et al. (T. Riedel, Dissertation University of Karsruhe 2002; H. Schulz et al., Appl. Catal. A, 186 (1999) 215; T. Riedel et al., Appl. Catal. A, 186 (1999) 201; T. Riedel et al., Ind. Eng. Chem. Res., 40 (2001) 1355; H. Schulz et al., Topics Catal., 32 (2005) 117) deal with the hydrogenation of CO₂ by Fe/Al₂O₃ catalysts which contain the promoters copper and potassium. Furthermore, in T. Riedel et al., Appl. Catal. A, 186 (1999) 201, in addition to iron catalysts on Al₂O₃ carriers, those on TiO₂ or SiO₂ carriers for the hydrogenation of CO₂ are also described. In G. Kishan et al., Catal. Lett. 56 (1998) 215 Fe—K on Al₂O₃, MgO or Al₂O₃ MgO with different portions of Al₂O₃ and MgO was used in the hydrogenation of CO₂. Iron catalysts, which can contain cerium as a promoter, were applied to alkali metal ion-exchanged zeolites and tested in the hydrogenation of CO₂ in S.-S. Nam et al., J. Chem. Res. (1999) 344 and S.-S. Nam et al., Appl. Catal. A 179 (1999) 155. A publication by R. W. Dorner et al. (R. W. Dorner et al., “Effects of Loading and Doping on Iron-based CO₂ Hydrogenation Catalysts”, Naval Research Laboratory, NRL/MR/6180-09-9200 (2009)) deals with CO₂ hydrogenation in the presence of iron catalysts supported on γ-Al₂O₃ which can be doped with manganese. The catalyst 100Fe6, 6Cu15, 7Al4K was tested in S.-R. Yan et al., Appl. Catal. A 194-195 (2000) 63-70 with regard to its activity in the hydrogenation of CO₂.

The not very extensive literature in the field of catalytic CO₂ hydrogenation is summarized in P. S. S. Prasad et al., Catal. Surv. Asia 12 (2008) 170.

It is the aim of the invention to provide a process for the production of hydrocarbons and a catalyst for this purpose by means of which high selectivity for hydrocarbons, in particular chain lengths C₅-C₁₅, but also chain lengths C₂-C₄, is achieved with the highest possible conversion of CO₂ without any recirculation of excess CO₂. In particular, a process is sought by means of which higher selectivity for liquid C₅-C₁₅ hydrocarbons, which are suitable for example as fuels, and a higher CO₂ conversion than with the catalysts of the prior art, for example 100Fe6, 6Cu15, 7Al4K of S.-R. Yan et al. in Appl. Catal. A 194-195 (2000) 63-70, are achieved.

### SUMMARY OF THE INVENTION

The object is achieved according to the invention by a process for producing hydrocarbons from carbon dioxide and hydrogen in the presence of a special catalyst, as defined in the attached claim 1. The catalyst used in the process according to the invention contains iron and at least one alkali metal and furthermore fulfils at least one of the following features (i) to (iii):

(i) in relation to the overall weight of the catalyst, the catalyst contains iron in an amount of at least 10% by weight, preferably of 15 and 35% by weight, and furthermore copper, and at least one additional representative selected from magnesium, zinc, lanthanum and zirconium;
(ii) the catalyst comprises TiO₂ and/or an aluminum magnesium hydroxycarbonate as a catalyst carrier;
iii) the catalyst also contains ruthenium and/or cobalt.

One particular embodiment of the above catalyst with feature (i) is the subject matter of the attached claim 13.

Advantageous further developments of the process according to the invention for producing hydrocarbons from carbon dioxide and hydrogen and of the catalyst according to the invention are the subject matter of the sub-claims.

### DETAILED DESCRIPTION OF THE INVENTION

In the catalyst according to the invention and in the catalyst of the process according to the invention the metals, such as iron, alkali metal (e.g. lithium, sodium and potassium), copper, magnesium, zinc, lanthanum, aluminum, zirconium, manganese, ruthenium and cobalt can also be present as compounds, in particular as oxides. Preferably, at least part of the metals, particularly preferably all of the metals, are present as oxide.

In the process according to the invention for producing hydrocarbons from carbon dioxide and hydrogen, carbon dioxide is converted with hydrogen in the presence of a catalyst, the catalyst containing iron and at least one alkali metal.

The at least one alkali metal is advantageously selected from the group consisting of lithium, sodium, potassium and rubidium. Preferably, the catalyst contains potassium as the at least one alkali metal. Preferably, the portion of the at least one alkali metal is 1-30% by weight and particularly preferably 5-12% by weight in relation to the iron portion in the catalyst. If the catalyst in the process according to the invention contains potassium, the potassium content is preferably 1-30% by weight and particularly preferably 5-12% by weight in relation to the iron portion in the catalyst.

In the process according to the invention it is essential that the catalyst fulfils at least one of the following features (i) to (iii):

(i) the catalyst contains, in relation to the overall weight of the catalyst, iron in an amount of at least 10% by weight, preferably of 15 and 35% by weight, and furthermore copper and at least one other representative, selected from magnesium, zinc, lanthanum and zirconium;
(ii) the catalyst comprises TiO₂ and/or an aluminum magnesium hydroxycarbonate as a catalyst carrier;
(iii) furthermore, the catalyst contains ruthenium and/or cobalt.

One embodiment of the process according to the invention relates to the use of a catalyst which fulfils feature (i) (process (i)).

Preferably, the at least one alkali metal that is contained in the catalyst of process (i) is potassium. Optionally, the catalyst of process (i) can contain two additional representatives selected from magnesium, zinc, lanthanum and zirconium. The catalyst of process (i) can, moreover, contain manganese and/or aluminum. For example, the catalyst of process (i) can contain manganese and copper. In preferred embodiments of the process according to the invention the catalyst contains:
potassium and lanthanum;
potassium and magnesium;
potassium and zirconium;
potassium and zinc;
potassium, magnesium and manganese; or
potassium, zirconium and manganese,
   and particularly preferably here:
potassium, magnesium and manganese; or
potassium, zirconium and manganese.

The components potassium, copper, magnesium, zinc, lanthanum, zirconium, manganese and aluminum constitute promoters here. For the purposes of the present application a promoter is defined as a constituent or component of a catalyst that has a positive effect upon the catalysis reaction.

The weight ratio of iron to the overall weight of all of the promoters in the catalyst of process (i) is preferably between 1.5 and 15.

Preferably, the catalyst of process (i) additionally contains ruthenium. The components iron and ruthenium constitute active components. For the purposes of the present application an active component is defined as the catalytically active constituent or component.

The catalyst of process (i) can comprise a catalyst carrier to which the active components and promoters are applied (supported catalyst).

If the catalyst of process (i) does not comprise a catalyst carrier (unsupported catalyst), the overall weight of all of the promoters in the catalyst in relation to the catalyst overall is preferably between 15 and 50% by weight and particularly preferably between 30 and 40% by weight. Particularly preferred examples of the unsupported catalyst of process (i) are:

### 11, 3La14, 1Cu70, 7Fe3, 9K; and

### 11, 6Mg11, 6Mn11, 6Cu58, 2Fe7, 0K,

the metals being able to be present as oxide, and the portions of the metals being specified in % by weight in relation to the overall weight of all of the metals in metallic form in the catalyst. Preferably, at least part of the metals, particularly preferably all of the metals, are present as oxide.

If the catalyst of method (i) is supported, the catalyst carrier can be selected from the group consisting of Al₂O₃, Al₂O₃ MgO, TiO₂, La₂O₃, ZrO₂, ZrO₂ La₂O₃, ZrO₂ SiO₂, zeolites and aluminum magnesium hydroxycarbonates. Al₂O₃ MgO indicates mixtures of Al₂O₃ and MgO, the mixture ratio being able to be arbitrary. ZrO₂ La₂O₃ indicates mixtures of ZrO₂ and La₂O₃, the mixture ratio being able to be arbitrary, and preferably the weight ratio of La₂O₃:ZrO₂ is between 5:95 and 15:85. ZrO₂ SiO₂ indicates mixtures of ZrO₂ and SiO₂, the mixture ratio being able to be arbitrary. In the aluminum magnesium hydroxycarbonates the weight ratio between Al₂O₃ and MgO can be arbitrary. Preferably, the weight ratio of Al₂O₃:MgO is between 30:70 (e.g. Pural MG70) and 50:50. The aluminum magnesium hydroxycarbonates comprise amorphous and crystalline forms, and of the crystalline forms hydrotalcite is preferred.

Preferred catalyst carriers are TiO₂, Al₂O₃ and aluminum magnesium hydroxycarbonate, TiO₂ being particularly preferred. The carrier materials Al₂O₃ and TiO₂ are advantageous with regard to the yield of C₅-C₁₅ hydrocarbons. If TiO₂ is used as a catalyst carrier in the catalyst of process (i), a high yield of C₅-C₁₅ hydrocarbons is achieved, while at the same time a small amount of methane is formed. The catalyst carrier preferably has a particle size of 100 to 250 μm.

The overall weight of all of the promoters in the catalyst of process (i) is preferably between 1.5 and 20% by weight and particularly preferably between 3 and 10% by weight in relation to the weight of the catalyst overall.

The supported catalyst of process (i) is preferably selected from the following catalysts:

### 0.07Ru 1.27Mg 1.78Cu 20Fe 1.74K/Al₂O₃;

### 0.1Ru 0.26Mg 20Fe 2.1K/Al₂O₃;

### 0.08Ru 0.95Mg 2.49Cu 20Fe 1.52K/Al₂O₃;

### 0.17Ru 1.15Mg 2.76Cu 20Fe 1.82K/Al₂O₃;

### 0.17Ru 1.15Mg 2.19Cu 20Fe 1.52K/Al₂O₃;

### 0.02Ru 1.02Mg 0.1Zr 20Fe 0.83K/Al₂O₃;

### 0.63La 4Cu 20Fe 1.31K/Al₂O₃;

### 0.07Ru 0.3Zn 20Fe 2.1K/TiO₂;

### 2.23Zr 4Cu 20Fe 1.73K/TiO₂;

### 0.1Ru 0.2Cu 0.29Zn 20Fe 1.4K/TiO₂;

### 1.01 Cu 20Fe 1.7K/TiO₂;

### 2.3Zr 1.8Mn 2.6Zn 20Fe 1.7K/TiO₂;

### 3.27Zr 2.96Mn 1Cu 20Fe 1.48K/TiO₂;

### 20Fe 2.66Al 2.14Cu 1.78K/TiO₂;

### 3.18La 4Cu 20Fe 1.1K/TiO₂;

20Fe 1.78Cu 1.74K 1.27Mg 0.07Ru/aluminum magnesium hydroxycarbonate;
20Fe 4Mg 4Mn 4Cu 2.4K/aluminum magnesium hydroxycarbonate;
and
20Fe 2.1K 0.3Zn 0.07Ru/aluminum magnesium hydroxycarbonate, the metals being able to be present as oxide and the portions of the metals being specified in % by weight in relation to the total of the overall weight of all of the metals in metallic form in the catalyst and of the catalyst carrier. Preferably at least part of the metals, particularly preferably all of the metals, are present as oxide.

Another embodiment of the process according to the invention relates to the use of a catalyst that fulfils feature (ii) (process ii)). If TiO₂ or aluminum magnesium hydroxycarbonate is used in the catalyst of the process according to the invention as a catalyst carrier, a particularly high yield of valuable hydrocarbons, such as for example C₂-C₄ and C₅-C₁₅ hydrocarbons is achieved, while at the same time a small amount of undesired methane is formed. The catalyst carriers TiO₂ and aluminum magnesium hydrocarbonates preferably have a particle size of 100 to 250 μm.

The portion of iron in the catalyst, in relation to the catalyst overall, is preferably between 10 and 50% by weight iron, particularly preferably between 15 and 35% by weight iron and very particularly preferably between 20 and 25% by weight iron. By means of this high portion of iron, a particularly high CO₂ conversion is achieved.

Preferably, the catalyst in process (ii) contains potassium as the at least one alkali metal. In one preferred embodiment of process (ii) the catalyst contains at least one other representative, the latter being selected from the group consisting of magnesium, zinc, lanthanum, aluminum, zirconium, manganese and copper. Optionally, two, three or four other representatives can be contained in the catalyst of process (ii). Preferably, the at least one other representative is magnesium, zinc or copper. Particularly preferably, the catalyst in process (ii) contains potassium and copper and at least one other representative that is selected from the group consisting of aluminum, lanthanum, zirconium, zinc and magnesium. In preferred embodiments of process (ii) the catalyst contains:
potassium and zinc;
potassium and magnesium;
potassium and copper;
potassium, copper and zirconium;
potassium, copper and zinc;
potassium, copper and aluminum;
potassium, copper and lanthanum;
potassium, copper, magnesium and manganese;
potassium, zirconium, manganese and zinc; or
potassium, copper, zirconium and manganese,
   and particularly preferred here are:
potassium, zirconium, manganese and zinc; or
potassium, copper, zirconium and manganese.

The components potassium, copper, magnesium, zinc, lanthanum, zirconium, manganese and aluminum constitute promoters here.

The weight ratio of iron to the overall weight of all of the promoters in the catalyst of process (ii) is preferably between 1.5 and 15.

The overall weight of all of the promoters in the catalyst of process (ii) is preferably between 1.5 and 20% by weight and particularly preferably between 3 and 10% by weight in relation to the weight of the catalyst overall.

Preferably, the catalyst of process (ii) additionally contains ruthenium. The components iron and ruthenium constitute active components.

Examples of the catalysts of process (ii) are:

### 0.07Ru 0.3Zn 20Fe 2.1K/TiO₂;

### 0.2Ru 20Fe 1.99K/TiO₂;

### 0.2Ru 20Fe 1.52K/TiO₂;

### 2.23Zr 4Cu 20Fe 1.73K/TiO₂;

### 0.1Ru 0.2Cu 0.29Zn 20Fe 1.4K/TiO₂;

### 0.01Cu 20Fe 1.7K/TiO₂;

### 2.3Zr 1.8 Mn 2.6Zn 20Fe 1.7K/TiO₂;

### 0.2Ru 20Fe 0.98K/TiO₂;

### 3.27Zr 2.96Mn 1Cu 20Fe 1.48K/TiO₂;

### 0.2Ru 20Fe 1.16K/TiO₂;

### 20Fe 2.66Al 2.14Cu 1.78K/TiO₂; and

### 3.18La 4Cu 20Fe 1.1K/TiO₂,

the metals being able to be present as oxide, and the portions of the metals being specified in % by weight, in relation to the sum of the overall weight of all of the metals in metallic form in the catalyst and of the catalyst carrier. Preferably, at least part of the metals, and particularly preferably all of the metals are present as oxide.

Another embodiment of the process according to the invention relates to the use of a catalyst which fulfils feature (iii) (process iii)). In this embodiment the components iron, ruthenium and cobalt constitute active components.

Preferably, the catalyst of process (iii) contains potassium as the at least one alkali metal. In a preferred embodiment of process (iii) the catalyst contains at least one other representative, the latter being selected from the group consisting of magnesium, zinc, lanthanum, aluminum, zirconium, manganese and copper. Optionally, two, three or four additional representatives can be contained in the catalyst of process (iii). Preferably, the at least one additional representative is magnesium, zinc or copper. Particularly preferably the catalyst in process (iii) contains potassium and copper and at least one other representative that is selected from the group consisting of aluminum, lanthanum, zirconium, zinc and magnesium. In particularly preferred embodiments of process (iii) the catalyst contains:
potassium and zinc;
potassium and magnesium;
potassium and copper;
potassium, zirconium, manganese and zinc;
potassium, copper, magnesium and manganese; or
potassium, copper, zirconium and manganese.

The components potassium, copper, magnesium, zinc, lanthanum, zirconium, manganese and aluminum constitute promoters here.

The weight ratio of iron to the overall weight of all of the promoters in the catalyst of process (iii) is preferably between 1.5 and 15.

The catalyst in process (iii) can be both supported and unsupported.

If the catalyst of method (iii) is unsupported, the overall weight of all of the promoters in the catalyst in relation to the catalyst overall is preferably between 15 and 50% by weight and particularly preferably between 30 and 40% by weight.

If the catalyst of process (iii) is supported—this is a preferred embodiment—the catalyst carrier can be selected from the group consisting of Al₂O₃, Al₂O₃ MgO, TiO₂, La₂O₃, ZrO₂, ZrO₂ La₂O₃, ZrO₂ SiO₂, zeolites and aluminum magnesium hydroxycarbonates. Al₂O₃ MgO indicates mixtures of Al₂O₃ and MgO, the mixture ratio being able to be arbitrary. ZrO₂ La₂O₃ indicates mixtures of ZrO₂ and La₂O₃, the mixture ratio being able to be arbitrary, and preferably the weight ratio of La₂O₃:ZrO₂ is between 5:95 and 15:85. ZrO₂ SiO₂ indicates mixtures of ZrO₂ and SiO₂, the mixture ratio being able to be arbitrary. In the aluminum magnesium hydroxycarbonates the weight ratio between Al₂O₃ and MgO can be arbitrary. Preferably, the weight ratio of Al₂O₃:MgO is between 30:70 (e.g. Pural MG70) and 50:50. The aluminum magnesium hydroxycarbonates comprise amorphous and crystalline forms, and of the crystalline forms, hydrotalcite is preferred.

Preferred catalyst carriers are TiO₂, Al₂O₃ and aluminum magnesium hydroxycarbonate, TiO₂ being particularly preferred. These carrier materials are advantageous with regard to the yield of C₅-C₁₅ hydrocarbons. The catalyst carrier preferably has a particle size of 100 to 250 μm.

The overall weight of all of the promoters in the catalyst of process (iii) is preferably between 1.5 and 20% by weight, and particularly preferably between 3 and 10% by weight in relation to the weight of the catalyst overall.

In process (iii) the following catalyst is particularly preferred:

### 0.2Ru 18.08Fe 2.17K 1.92Co/Al₂O₃,

the metals being able to be present as oxide and the portions of the metals being specified in % by weight in relation to the sum of the overall weight of all of the metals in metallic form in the catalyst and of the catalyst carrier. Preferably, at least part of the metals, particularly preferably all of the metals are present as oxide.

Another embodiment of the process according to the invention (process (iv)) relates to the production of hydrocarbons from carbon dioxide and hydrogen wherein carbon dioxide is converted with hydrogen in the presence of a catalyst, the catalyst containing iron, potassium, copper and magnesium, iron being contained in an amount of at least 8% by weight, preferably between 10 and 50% by weight, particularly preferably between 15 and 35% by weight in relation to the overall weight of the catalyst. Moreover, the catalyst can contain manganese. Preferably, the portion of potassium is 1-30% by weight and particularly preferably 5-12% by weight in relation to the iron portion in the catalyst. The components potassium, copper, magnesium and manganese constitute promoters here. Iron is contained as an active component.

The weight ratio of iron to the overall weight of all of the promoters in the catalyst of the process is preferably between 1.5 and 15.

The catalyst of the process can comprise a catalyst carrier to which the active components and promoters are applied (supported catalyst).

If the catalyst of the process does not comprise a catalyst carrier (unsupported catalyst), the overall weight of all of the promoters in the catalyst in relation to the catalyst overall is preferably between 15 and 50% by weight and particularly preferably between 30 and 40% by weight.

If the catalyst of the process is supported, the catalyst carrier can be selected from the group consisting of Al₂O₃, Al₂O₃ MgO, TiO₂, La₂O₃, ZrO₂, ZrO₂ La₂O₃, ZrO₂ SiO₂, zeolites and aluminum magnesium hydroxycarbonates. Al₂O₃ MgO indicates mixtures of Al₂O₃ and MgO, the mixture ratio being able to be arbitrary. ZrO₂ La₂O₃ indicates mixtures of ZrO₂ and La₂O₃, the mixture ratio being able to be arbitrary, and preferably the weight ratio of La₂O₃:ZrO₂ is between 5:95 and 15:85. ZrO₂ SiO₂ indicates mixtures of ZrO₂ and SiO₂, the mixture ratio being able to be arbitrary. In the aluminum magnesium hydroxycarbonates the weight ratio between Al₂O₃ and MgO can be arbitrary. Preferably, the weight ratio of Al₂O₃:MgO is between 30:70 (e.g. Pural MG70) and 50:50. The aluminum magnesium hydroxycarbonates comprise amorphous and crystalline forms, and of the crystalline forms hydrotalcite is preferred.

The catalyst carrier is preferably Al₂O₃. The catalyst carrier preferably has a particle size of 100 to 250 μm.

The overall weight of all of the promoters in the catalyst of the process is preferably between 1.5 and 20% by weight and particularly preferably is between 3 and 10% by weight in relation to the weight of the catalyst overall.

The following catalysts are particularly preferred:

### 10Fe 1.0Mg 2.0Cu 0.8K/Al₂O₃

### 10 Fe 2.0Mg 2.0Mn 2.0Cu 1.2K/Al₂O₃,

the metals being able to be present as oxide and the portions of the metals being specified in % by weight in relation to the sum of the overall weight of all of the metals in metallic form in the catalyst and of the catalyst carrier. Preferably, at least part of the metals, and particularly preferably all of the metals, are present as oxide.

Another aspect of the invention relates to the use of the catalysts, as described above, in the process according to the invention, as defined, for example, in the attached Claim 1.

In the following the process according to the invention is described in general. The process according to the invention relates to the conversion of carbon dioxide with hydrogen in the presence of a catalyst, i.e. the hydrogenation of carbon dioxide by hydrogen in the presence of a catalyst to form hydrocarbons. Here the RWGS reaction of carbon dioxide to form carbon monoxide preferably takes place, and the carbon monoxide that is formed is converted in a Fischer-Tropsch synthesis into hydrocarbons. Therefore, the process according to the invention can also be generally called a Fischer-Tropsch process. Preferably, the hydrocarbons are C₅-C₁₅ hydrocarbons and/or C₂-C₄ hydrocarbons.

The conversion of the carbon dioxide with hydrogen in the process according to the invention preferably takes place at a temperature of 150 to 400° C., particularly preferably at 300 to 370° C., even more preferably at 320° C. to 370° C. and very particularly preferably at 350° C. A temperature of 150 to 400° C. leads to a particularly high yield of hydrocarbons, in particular C₅-C₁₅ hydrocarbons. A temperature of 300° C. to 350° C. is particularly suitable in relation to the yield of C₅-C₁₅ hydrocarbons. In one preferred embodiment of the process according to the invention the conversion of the carbon dioxide with hydrogen takes place at a pressure of 10 to 50 bar, and particularly preferably between 15 and 30 bar. Preferably, the conversion of the carbon dioxide with hydrogen takes place at a temperature of 300° C. and a pressure of 15 bar or at a temperature of 350° C. and a pressure of 10 bar.

In a preferred embodiment of the process according to the invention, the molar ratio of the hydrogen used to carbon dioxide is 4 to 8 and preferably 6.

The carbon dioxide used in the process according to the invention can be obtained by the separation of flue gas from the exhaust gas of power stations which preferably generate electrical energy from fossil fuels. The hydrogen gas used in the process according to the invention can be obtained by electrolysis processes, the latter preferably being operated by current from photovoltaic or wind power plants or other CO₂-neutral facilities.

The process according to the invention for the production of hydrocarbons from carbon dioxide and hydrogen can be carried out in a fixed bed, fluidized bed or bubble column reactor. Examples of suitable fixed bed reactors are catalytic fixed bed reactors, bubble column reactors and tube bundle reactors through which gas flows. In the process according to the invention a catalytic fixed bed reactor through which gas flows is preferably used.

Obtained in the conversion of carbon dioxide and hydrogen in the process according to the invention is a product mixture which may contain the hydrocarbons, non-converted hydrogen, water, non-converted carbon dioxide and carbon monoxide. The carbon monoxide is intermediately formed from carbon dioxide and hydrogen in the RWGS. Preferably, the product mixture contains C₅-C₁₅ and C₂-C₄ hydrocarbons. If the C₂-C₄ hydrocarbons contain C₂-C₄ alkanes, the latter can be dehydrogenated after their separation to form C₂-C₄ alkenes, in particular ethylene, propylene and butenes.

C₅-C₁₅ hydrocarbons comprise linear, branched and cyclic alkanes and alkenes with a chain length of 5 to 15 carbon atoms. Examples of C₅-C₁₅ alkanes are linear, branched and cyclic isomers of pentanes, hexanes, heptanes, octanes, nonanes, decanes, undecanes, dodecanes, tridecanes, tetradecanes and pentadecanes. Examples of C₅-C₁₅ alkenes comprise linear, branched and cyclic isomers of pentenes, hexenes, heptenes, octenes, nonenes, decenes, undecenes, dodecenes, tridecenes, tetradecenes and pentadecenes. Preferably, the C₅-C₁₅ hydrocarbons are linear and/or branched alkanes and alkenes.

C₂-C₄ hydrocarbons comprise C₂-C₄ alkanes and C₂-C₄ alkenes which contain 2 to 4 carbon atoms, the C₄ alkanes and C₄ alkenes being able to be both linear and branched. Examples of C₂-C₄ hydrocarbons are ethane, ethylene, propane, propylene, n-butane, iso-butane, 1-butene, 2-butene and 2-methyl-1-propene. Butenes comprise n-butane, iso-butane, 1-butene, 2-butene and 2-methyl-1-propene.

With the process according to the invention it is possible to produce predominantly the valuable C₂-C₁₅ hydrocarbons from CO₂. Accordingly, in the process according to the invention the hydrocarbons produced are preferably predominantly C₂-C₁₅ hydrocarbons, i.e. the hydrocarbons produced comprise C₂-C₁₅ hydrocarbons in a portion of at least 50 mol %. In other words, the hydrocarbons of the product mixture obtained in the process according to the invention consist preferably by ≧50 mol %, and more preferably by >70 mol % of C₂-C₁₅ hydrocarbons in relation to the hydrocarbons produced.

With the process according to the invention it is in particular possible to obtain a greater molar portion of C₅-C₁₅ hydrocarbons (which can be used for example as fuels such as benzine, diesel and kerosene) than of C₂-C₄ hydrocarbons (which optionally serve, after dehydrogenation, as starting materials for the chemical industry). In other words, the molar ratio of C₅-C₁₅ hydrocarbons to C₂-C₄ hydrocarbons in the product mixture is preferably >1.

In the process according to the invention methane and higher hydrocarbons (with more than 16 carbon atoms), in particular waxes, are obtained as by-products. These by-products can be used in the conventional manner. For example, the methane produced in the process according to the invention can be used for heating purposes.

The product gas is preferably cooled to 35° C. in a phase separator so as to thereby separate out water in liquid form. If necessary, in a further step excess carbon dioxide can be removed from the product gas by absorption. For this purpose the product gas can be conveyed by a medium, for example a solvent, that absorbs carbon dioxide. Alternatively, the carbon dioxide can be removed from the product gas by pressure swing absorption (PSA adsorption) or by means of a means of a membrane. The separated out carbon dioxide gas can be delivered to the flow of reagent gas which comprises hydrogen gas and carbon dioxide gas in the process according to the invention.

In a low temperature separator or by means of a low temperature separating technique the product gas can be separated into the components that are liquid or gaseous at room temperature (20° C.), a low temperature heat exchanger being preferred. The C₅-C₁₅ hydrocarbons are obtained as liquids, whereas the C₂-C₄ hydrocarbons, methane, excess carbon monoxide and hydrogen are obtained gaseously.

Optionally, excess hydrogen gas can be recycled by methane being removed from the gas mixture of methane, excess carbon monoxide and hydrogen by means of membrane separating technology or low temperature separation. The recycled hydrogen gas can then be delivered back to the reagent gas flow of the process according to the invention.

In a preferred embodiment of the process according to the invention the catalyst is reduced with hydrogen before use in the process according to the invention. For this purpose the catalyst is treated, for example at 500° C., in a flow of inert gas (e.g. nitrogen) that contains hydrogen.

The catalyst that is used in the process according to the invention can be produced by precipitation from metal salt solutions or by milling metal oxides. In this way one obtains an unsupported catalyst. A supported catalyst can be produced by a catalyst carrier being impregnated, for example soak-impregnated, with metal salt solutions, e.g. aqueous metal nitrate solutions. One preferably starts here with the solution which has the highest concentration (e.g. in g/l). Preferably, the impregnated catalyst carrier is dried after each impregnation step. Preferably, oxalate is added to the catalysts during production, the oxalate preferably being added in the form of ammonium oxalate or metal oxalate. Ammonium oxalate is particularly preferred. As has been shown, this leads to better distribution of the active components, in particular of iron, and of the promoters in the catalyst or on the carrier.

The precipitated or ground metal salts and the impregnated catalyst carriers are preferably calcinated after drying. The calcination can be carried out, for example, in a muffle furnace in stagnant air (e.g. at 350° C.) or in the inert gas flow (e.g. at 400° C.). If a catalyst that contains oxalate is calcinated, total decomposition (combustion) of the oxalate takes place, i.e. the oxalate is removed without any residue by the calcination.

According to another aspect, the invention relates to the catalyst as such. The term “catalyst” makes it clear here that this is a composition or compound that is suitable as a catalyst, in particular in the process according to the invention, for producing hydrocarbons.

The catalyst according to the invention comprises as catalyst components, in relation to the overall weight of the catalyst, at least 10% by weight, preferably 10-50% by weight, particularly preferably 15 and 35% by weight and very particularly preferably 20-25% by weight iron as well as potassium, copper and at least one other component selected from magnesium, zinc, lanthanum and zirconium. In addition, the catalyst according to the invention can comprise at least one of the following metals: manganese, ruthenium, aluminum and cobalt.

Preferred embodiments of the catalyst according to the invention contain
iron, potassium, copper and lanthanum;
iron, potassium, copper and zirconium;
iron, potassium, copper, magnesium and manganese;
iron, potassium, copper, zirconium and manganese;
iron, ruthenium, potassium, copper and magnesium; or
iron, ruthenium, potassium, copper and zinc.

The components iron and ruthenium constitute active components.

The weight ratio of iron to the overall weight of all of the metals (promoters) from the group potassium, copper, magnesium, zinc, lanthanum, aluminum, zirconium and manganese in the catalyst according to the invention is preferably between 1.5 and 15. Preferably, at least part of the metals, particularly preferably all of the metals, are present as oxide.

The catalyst according to the invention can be both supported and unsupported.

If the catalyst according to the invention is unsupported, the overall weight of all of the metals from the group potassium, copper, magnesium, zinc, lanthanum, aluminum, zirconium and manganese in the catalyst in relation to the catalyst as a whole is preferably between 15 and 50% by weight and particularly preferably between 30 and 40% by weight. Particularly preferred examples of the unsupported catalyst of the invention are:

### 11, 3La14, 1Cu70, 7Fe3, 9K; and

### 11, 6Mg 11, 6Mn 11, 6Cu58, 2Fe7, 0K,

the metals being able to be present as oxide, and the portions of the metals being specified in % by weight in relation to the overall weight of all of the metals in metallic form in the catalyst. Preferably, at least part of the metals, particularly preferably all of the metals, are present as oxide.

If the catalyst according to the invention is supported, the carrier can be selected from the group consisting of Al₂O₃, Al₂O₃ MgO, TiO₂, La₂O₃, ZrO₂, ZrO₂ La₂O₃, ZrO₂ SiO₂, zeolites and aluminum magnesium hydroxycarbonates. Al₂O₃ MgO indicates mixtures of Al₂O₃ and MgO, the mixture ratio being able to be arbitrary. ZrO₂ La₂O₃ indicates mixtures of ZrO₂ and La₂O₃, the mixture ratio being able to be arbitrary, and preferably the weight ratio of La₂O₃:ZrO₂ is between 5:95 and 15:85. ZrO₂ SiO₂ indicates mixtures of ZrO₂ and SiO₂, the mixture ratio being able to be arbitrary. In the aluminum magnesium hydroxycarbonates the weight ratio between Al₂O₃ and MgO can be arbitrary. Preferably, the weight ratio of Al₂O₃:MgO is between 30:70 (e.g. Pural MG70) and 50:50. The aluminum magnesium hydroxycarbonates comprise amorphous and crystalline forms, and of the crystalline forms hydrotalcite is preferred.

Preferred catalyst carriers are TiO₂, Al₂O₃ and aluminum magnesium hydroxycarbonate. These carrier materials are advantageous with regard to the yield of C₅-C₁₅ hydrocarbons. If the catalyst contains TiO₂ as a carrier, a high yield of C₅-C₁₅ hydrocarbons will be achieved in the conversion of carbon dioxide with hydrogen in the presence of the catalyst, while at the same time a small amount of methane is formed.

The overall weight of all of the metals from the group potassium, copper, magnesium, zinc, lanthanum, aluminum, zirconium and manganese in the supported catalyst is preferably between 1.5 and 20% by weight, and particularly preferably between 3 and 10% by weight in relation to the weight of the catalyst overall.

The supported catalyst of the invention is preferably selected from the following catalysts:

### 0.07Ru 1.27 Mg 1.78Cu 20Fe 1.74K/Al₂O₃;

### 0.08Ru 0.91Mg 2.39Cu 19.23Fe 1.46K/Al₂O₃;

### 0.16Ru 1.11Mg 2.66Cu 19.26Fe 1.75K/Al₂O₃;

### 0.16Ru 1.11Mg 2.11Cu 19.23Fe 1.46K/Al₂O₃;

### 20Fe 2.66Al 2.14Cu 1.78K/TiO₂;

### 3.18La 4Cu 20Fe 1.1K/TiO₂;

### 0.63La 4Cu 20Fe 1.31K/Al₂O₃;

### 2.23Zr 4Cu 20Fe 1.73K/TiO₂;

### 0.1Ru 0.2Cu 0.29Zn 20Fe 1.4K/TiO₂;

### 3.27Zr 2.96Mn 1Cu 20Fe 1.48K/TiO₂;

20Fe 1.78Cu 1.74K 1.27Mg 0.07Ru/aluminum magnesium hydroxycarbonate; and
20Fe 4Mg 4Mn 4Cu 2.4K/aluminum magnesium hydroxycarbonate,
the metals being able to be present as oxide, and the portions of the metals being specified in % by weight in relation to the sum of the overall weight of all of the metals in metallic form in the catalyst and of the carrier. Preferably, at least part of the metals, particularly preferably all of the metals, are present as oxide.

Another aspect of the invention relates to the use of the catalysts, as described above, in any of processes (i) to (iv) according to the invention.

As is normal, in the aforementioned catalysts the part following the symbol “/” designates the carrier. All of the components before “/” represent the active components and promoters, the latter being able to be at least partially present in oxidic form. For example, 0.07Ru 0.3Zn 20Fe 1.1K/TiO₂ means that the catalyst contains TiO₂ as a carrier, and 0.07% by weight Ru, 0.3% by weight Zn, 20% by weight Fe and 1.1% by weight K, all in relation to the sum of the overall weight of all of the metals in metallic form in the catalyst and of the catalyst carrier.

In the following examples are given which describe the invention in more detail, but which are not intended to be restrictive.

### Examples

### Catalyst 1: 11.3La 14.1Cu 70.7Fe 3.9K

0.71 g La (NO₃)₃.6H₂O, 1.07 g Cu(NO₃)₂.3H₂O, 9.49 g Fe(NO₃)₃.9H₂O and 0.20 g KNO₃ were dissolved in 100 ml dist. water and heated to 90° C. A 25% NH₃ solution was dripped into the warm solution until no more precipitation was to be observed. Next the mixture was evaporated slowly to dryness, dried in the dry cabinet at 130° C. and calcinated in the muffle furnace at 350° C. for 5 hours.

### Catalyst 2: 11.6Mg 11.6Mn 11.6Cu 58.2Fe 7.0K

Catalyst 2 was produced in the same way as the production of catalyst 1 from 2.45 g Mg(NO₃)₂.6H₂O, 1.06 g Mn(NO₃)₂.4H₂O, 0.88 g Cu(NO₃)₂.3H₂O, 7.81 g Fe(NO₃)₃.9H₂O and 0.36 g KNO₃.

### Catalyst 3: 19.3Fe 1.5K 0.2Ru/TiO₂

Step 1: 5 ml of an aqueous Fe(NO₃)₃ solution (content 48.7 g/l Fe) was pipetted onto 1 g TiO₂ (Degussa Aerolyst 7708, particle size 0.1-0.25 mm) while shaking at room temperature, the mixture was heated to 100° C. and was evaporated at 100° C. while shaking and dried.

Step 2: 5 ml of an aqueous KNO₃ solution (content 3.691 g/l K) was pipetted onto the dried mixture from step 1 while shaking at room temperature, the mixture was heated to 100° C. and evaporated at 100° C. while shaking and dried.

Step 3: 5 ml of an aqueous Ru(NO) (NO₃)₃ solution (content 0.49 g/l Ru) was pipetted onto the dried mixture of step 2 while shaking at room temperature, the mixture was heated to 100° C. and and was evaporated at 100° C. while shaking and dried.

Step 4: The dried mixture was calcinated for 5 hours in the muffle furnace at 350° in air.

### Catalyst 4: 19.2 Fe 1.9K 0.2Ru/TiO₂

Catalyst 4 was produced in the same way as the production of catalyst 3, in step 2 the content of the aqueous KNO₃ solution being 4.85 g/l K.

### Catalyst 5: 0.1Ru 0.2Cu 0.3Zn 19.2Fe 1.3K/TiO₂

Catalyst 5 was produced in the same way as the production of catalyst 3, in step 1 the content of the aqueous Fe(NO₃)₃ solution being 48.8 g/l Fe, in step 2 the content of the aqueous KNO₃ solution being 3.41 g/l K, in step 3 the content of the aqueous Zn(NO₃)₂ solution being 0.71 g/l Zn and step 4 being replaced by the following steps 4 to 6:

Step 4: 5 ml of an aqueous Cu(NO₃)₂ solution (content 0.48 g/l Cu) was pipetted onto the dried mixture from step 3 while shaking at room temperature, the mixture was heated to 100° C. and evaporated at 100° C. while shaking and dried.

Step 5: 5 ml of an aqueous Ru(NO) (NO₃)₃ solution (content 0.26 g/l Ru) was pipetted onto the dried mixture of step 4 while shaking at room temperature, the mixture was heated to 100° C. and was evaporated at 100° C. while shaking and dried.

Step 6: The dried mixture was calcinated for 5 hours in the muffle furnace at 350° in air.

### Catalyst 6: 0.07Ru 0.3Zn 19.2Fe 2.0K/TiO₂

Catalyst 6 was produced in the same way as the production of catalyst 3, in step 1 the content of the aqueous Fe(NO₃)₃ solution being 49.0 g/l Fe, in step 2 the content of the aqueous KNO₃ solution being 5.13 g/l K, in step 3 the content of the aqueous Zn(NO₃)₂ solution being 0.73 g/l Zn and step 4 being replaced by the following steps 4 and 5:

Step 4: 5 ml of an aqueous Ru (NO) (NO₃)₃ solution (content 0.18 g/l Ru) was pipetted onto the dried mixture from step 3 while shaking at room temperature, the mixture was heated to 100° C. and evaporated at 100° C. while shaking and dried.

Step 5: The dried mixture was calcinated for 5 hours in the muffle furnace at 350° in air.

### Catalyst 7: 2.2Zr 1.7Mn 2.4Zn 18.8Fe 1.6K/TiO₂

Catalyst 7 was produced in the same way as the production of catalyst 5, in step 1 the content of the aqueous Fe(NO₃)₃ solution being 51.3 g/l Fe, in step 2 the content of the aqueous KNO₃ solution being 4.37 g/l K, in step 3 the content of the aqueous Zn(NO₃)₂ solution being 6.68 g/l Zn, step 4 the content of the aqueous ZrO(NO₃)₂ solution being 5.91 g/l Zr, and in step 5 the content of the aqueous Mn(NO₃)₂ solution being 4.62 g/l Mn.

### Catalyst 8: 0.97Cu 19.16Fe 1.63K/TiO₂

Catalyst 8 was produced in the same way as the production of catalyst 3, in step 1 5 ml of an aqueous solution of Fe(NO₃)₃ (content 49.1 g/l Fe) and 2 ml of an ammonium oxalate solution (content 25.15 g/l oxalate) being used, in step 2 the content of the aqueous KNO₃ solution being 4.17 g/l K and in step 3 the content of the aqueous Cu(NO₃)₂ solution being 2.48 g/l Cu.

### Catalyst 9: 2.1Zr 3.8Cu 18.8Fe 1.6K/TiO₂

Catalyst 9 was produced in the same way as the production of catalyst 6, in step 1 the content of the aqueous Fe(NO₃)₃ solution being 51.2 g/l Fe, in step 2 the content of the aqueous KNO₃ solution being 4.42 g/l K, in step 3 the content of the aqueous Cu(NO₃)₂ solution being 10.24 g/l Cu, and in step 4 the content of the aqueous ZrO(NO₃)₂ solution being 5.71 g/l Zr.

### Catalyst 10: 3.1Zr 2.8Mn 0.9Cu 18.8Fe 1.4K/TiO₂

Catalyst 10 was produced in the same way as the production of catalyst 5, in step 1 the content of the aqueous Fe(NO₃)₃ solution being 51.5 g/l Fe, in step 2 the content of the aqueous KNO₃ solution being 3.80 g/l K, in step 3 the content of the aqueous Zn(NO₃)₂ solution being 8.43 g/l Zr, in step 4 the content of the aqueous Mn(NO₃)₂ solution being 7.62 g/l Mn, and in step 5 the content of the aqueous Cu(NO₃)₂ solution being 2.57 g/l Cu.

### Results of the Catalytic Tests of Catalysts 1-10

Catalysts 1-10 were tested for CO₂ hydrogenation into hydrocarbons. For this purpose 0.3 g of a catalyst were respectively inserted into a stainless steel reactor (5 mm inside diameter). Before the start of the test the catalyst was reduced in a 10% H₂/N₂ flow for 2 hrs at 500° C. and 10 bar and then cooled in this gas to reaction temperature. The catalytic test took place under the reaction conditions specified in Table 1. The product mixture was analyzed gas chromatographically, and the conversions (X) and yields (Y) were calculated—reagent based—and the selectivity (S) was calculated—product-based.

The testing of the catalytic properties of catalysts 1-10 in relation to the hydrogenation of carbon dioxide with hydrogen took place in a fixed bed reactor (stainless steel pipe, inside diameter 5 mm). All of the catalysts were used in a grain fraction of 100-250 μm so as to minimize the effects of material transportation and to guarantee plug flow in the reactor.

The product gas was analyzed by on-line coupled gas chromatography in the gas phase. For this purpose an operating pressure of 10 bar was chosen, and on the other hand all of the pipe lines and valves were heated after the reactor and the GC inlet system was heated to at least 150° C. In addition, a hot gas separator (150° C.) and a cold gas separator (room temperature) were respectively integrated into the exhaust gas line with an integrated aerosol filter. Two-dimensional capillary gas chromatography was applied.

The following components were analyzed:
By means of a heat conductivity detector (HCD): CO₂, ethylene, ethane, water, H₂, N₂, methane, CO
By means of a flame ionization detector (FID): methane, C₂-C₄ (all alkane and olefin isomers), C₅-C₁₈ (n-alkanes and n−1 olefins), the other C₅-C₁₈ isomers were analyzed, but not structurally assigned.

An Agilent 7890 gas chromatograph with two independent channels was used for the analysis. Both channels were operated with helium as the carrier gas. One channel of the gas chromatograph was equipped with a Porapak Q column and a PoraPlot Q column. The columns were heated from the starting temperature of 50° C. to 200° C. with a temperature program. The second channel was equipped with an FFAP column and an Al₂O₃₁KCl column (25 m×0.32 mm 5 μm film thickness), the FFAP column being operated at a temperature of 50-200° C. The Al₂O₃/KCl column was operated with a temperature program of 80° C. to 200° C.

TABLE 1
Results of the catalytic test. Reaction conditions: p = 15 bar, H₂:CO₂ = 6, GHSV = 1320 ml g_{cat}⁻¹ h⁻¹

| | | | | | | | | | Olefinanteil |
|---|---|---|---|---|---|---|---|---|---|
| Katalysator | T in ° C. | X(CO₂) | X(H₂) | Y(C₅-C₁₅) | Y(C₂-C₄) | Y(CO) | Y(CH₄) | S(C₅-C₁₅) | (C₂-C₄) |
| 1 | 300 | 62% | 33% | 23% | 23% | 2% | 8% | 41% | 86% |
| 2 | 300 | 62% | 32% | 22% | 22% | 2% | 8% | 40% | 86% |
| 3 | 350 | 66% | 31% | 18% | 20% | 5% | 12% | 32% | 82% |
| 4 | 350 | 65% | 31% | 18% | 19% | 5% | 11% | 34% | 83% |
| 5 | 350 | 65% | 33% | 17% | 19% | 5% | 11% | 33% | 82% |
| 6 | 350 | 64% | 32% | 18% | 19% | 5% | 10% | 35% | 85% |
| 7 | 350 | 63% | 30% | 17% | 20% | 5% | 10% | 32% | 80% |
| 8 | 350 | 63% | 31% | 17% | 19% | 5% | 11% | 33% | 83% |
| 9 | 350 | 69% | 35% | 17% | 21% | 4% | 14% | 30% | 79% |
| 10 | 300 | 46% | 23% | 16% | 15% | 5% | 5% | 40% | 80% |

Katalysator = catalyst;
Olefinanteil = olefin portion
Olefin portion = Portion of C₂-C₄ olefins in mol % in relation to all of the C₂-C₄ hydrocarbons (Y(C₂-C₄))

### Catalysts 11-67

In the Sophas synthesis robot (Zinsser Analytics) catalysts 11-67 were produced by impregnating carrier materials. If a number of components are contained on one carrier, the impregnation was carried out sequentially, starting with the component with the highest concentration, after each impregnation step drying taking place at a temperature of 100° C. In this way it was ensured that all of the catalyst components were separated out on the carrier material.

After the final drying in the synthesis robot, the catalysts were calcinated in the muffle furnace for 5 hrs in stagnant air at 350° C. (heating rate 5 K/min). In contrast, the samples containing oxalate were heated at only 2.5 K/min due to the otherwise excessively fast oxalate decomposition.

### Starting Materials:

Carrier materials: γ-Al₂O₃ (Condea), TiO₂ (Degussa Aerolyst 7708), SiO₂ (Degussa Aerolyst 355), aluminum magnesium hydroxycarbonate with a weight ratio Al₂O₃:MgO of 30:70 (Pural MG70)

In Table 2 the compositions of catalysts 11-67 are summarized. The portion of the respective metal in metallic form is specified in % by weight and relates to the overall weight of all of the metals in metallic form in the catalyst and of the catalyst carrier.

TABLE 2
Compositions of catalysts 11-67

| | | Fe | Co | Ru | Al | La | Mg | Zr | Mn | Cu | Zn | K | |
|---|---|---|---|---|---|---|---|---|---|---|---|---|---|
| Katalysator | Träger | in % | in % | in % | in % | in % | in % | in % | in % | in % | in % | in % | Oxalat * |
| 11 | TiO₂ | 20 | | | | 3.2 | | | | 4.0 | | 1.1 | nein |
| 12 | TiO₂ | 20 | | 0.1 | | | | | | | 0.3 | 2.1 | nein |
| 13 | Al₂O₃ | 20 | | | | 0.6 | | | | 4.0 | | 1.3 | nein |
| 14 | Al₂O₃ | 18 | 2 | | | | 3.4 | | | 2.9 | | 1.6 | ja |
| 15 | Al₂O₃ | 17 | 3 | | | | 3.4 | | | 1.9 | | 1.6 | ja |
| 16 | TiO₂ | 20 | | | | 2.8 | | | | 4.0 | | 1.1 | nein |
| 17 | Al₂O₃ | 20 | | | | | | 2.8 | 1.9 | 3.0 | | 1.7 | ja |
| 18 | Al₂O₃ | 20 | | 0.1 | | | | | 0.1 | | | 1.5 | nein |
| 19 | Al₂O₃ | 18 | 2 | 0.2 | | | | | | | | 2.2 | nein |
| 20 | Al₂O₃ | 10 | | 0.1 | | | | | | 1.1 | 1.7 | 0.9 | ja |
| 21 | Al₂O₃ | 20 | | | | | | 2.0 | | 2.3 | | 1.7 | nein |
| 22 | TiO₂ | 10 | | | | | | | 1.3 | 1.7 | 1.1 | 0.7 | ja |
| 23 | TiO₂ | 10 | | | | | | | 2.0 | 2.0 | 2.0 | 0.6 | ja |
| 24 | Al₂O₃ | 10 | | 0.1 | | | | | | | | 1.2 | nein |
| 25 | Al₂O₃ | 10 | | 0.1 | | | | | | 2.0 | 1.9 | 0.8 | ja |
| 26 | Al₂O₃ | 10 | | | | | 1.0 | | | 2.0 | | 0.8 | ja |
| 27 | TiO₂ | 20 | | | | | | 2.2 | | 4.0 | | 1.7 | nein |

Katalysator = catalyst; Träger = carrier; Oxalat = oxalate; nein = no; ja = yes
*: Oxalate was used in the production of the catalyst, but oxalate is not contained in the composition of the calcinated catalyst.

| | | Fe | Co | Ru | Al | La | Mg | Zr | Mn | Cu | Zn | K | |
|---|---|---|---|---|---|---|---|---|---|---|---|---|---|
| Katalysator | Träger | in % | in % | in % | in % | in % | in % | in % | in % | in % | in % | in % | Oxalat * |
| 28 | Al₂O₃ | 20 | | | | 1.9 | | | | 3.7 | | 1.5 | ja |
| 29 | Al₂O₃ | 10 | | 0.1 | | | | | | | 2.0 | 1.2 | nein |
| 30 | TiO₂ | 20 | | | | 0.3 | | 2.5 | | 0.9 | | 1.7 | nein |
| 31 | TiO₂ | 20 | | | | | | | | 1.0 | | 1.7 | ja |
| 32 | TiO₂ | 20 | | | | | | 2.3 | 0.8 | 3.6 | | 1.1 | ja |
| 33 | TiO₂ | 20 | | | | | | 3.3 | 3.0 | 1.0 | | 1.5 | nein |
| 34 | TiO₂ | 10 | | 0.1 | | | | 1.0 | | 2.0 | | 1.2 | nein |
| 35 | TiO₂ | 20 | | 0.1 | | | | | | 2.1 | 0.1 | 1.0 | ja |
| 36 | TiO₂ | 20 | | | | | | 3.9 | | 1.0 | | 2.4 | nein |
| 37 | TiO₂ | 20 | | | | | | 2.3 | 1.8 | | 2.6 | 1.7 | nein |
| 38 | TiO₂ | 10 | | 0.1 | | | | | | | 2.0 | 1.2 | nein |
| 39 | Al₂O₃ | 10 | | | | | 2.0 | | 2.0 | 2.0 | | 1.2 | nein |
| 40 | TiO₂ | 20 | | 0.1 | | | | | | | 0.3 | 1.1 | nein |
| 41 | TiO₂ | 10 | | 0.1 | | | | 0.6 | | 2.0 | | 0.8 | nein |
| 42 | TiO₂ | 20 | | | | | | | | | 3.0 | 1.7 | ja |
| 43 | TiO₂ | 20 | | 0.07 | | | | | | | 0.3 | 2.1 | nein |
| 44 | TiO₂ | 20 | | | | | | 2.23 | | 4 | | 1.73 | nein |
| 45 | TiO₂ | 20 | | 0.07 | | | | | | | 0.3 | 1.1 | nein |

Katalysator = catalyst; Träger = carrier; Oxalat = oxalate; nein = no; ja = yes
*: Oxalate was used in the production of the catalyst, but oxalate is not contained in the composition of the calcinated catalyst.

| | | Fe | Co | Ru | Al | La | Mg | Zr | Mn | Cu | Zn | K | |
|---|---|---|---|---|---|---|---|---|---|---|---|---|---|
| Kat. | Träger | in % | in % | in % | in % | in % | in % | in % | in % | in % | in % | in % | Oxalat * |
| 46 | TiO₂ | 20 | | | | 0.32 | | 2.51 | | 0.88 | | 1.7 | nein |
| 47 | TiO₂ | 19.16 | | | | | | | | 0.97 | | 1.63 | nein |
| 48 | Al₂O₃ | 20 | | 0.07 | | | 1.27 | | | 1.78 | | 1.74 | nein |
| 49 | Al₂O₃ | 20 | | 0.1 | | | 0.26 | | | | | 2.1 | nein |
| 50 | Al₂O₃ | 19.23 | | 0.08 | | | 0.91 | | | 2.39 | | 1.46 | nein |
| 51 | Al₂O₃ | 19.26 | | 0.16 | | | 1.11 | | | 2.66 | | 1.75 | nein |
| 52 | Al₂O₃ | 19.23 | | 0.16 | | | 1.11 | | | 2.11 | | 1.46 | nein |
| 53 | TiO₂ | 20 | | | 2.66 | | | | | 2.14 | | 1.78 | nein |
| 54 | TiO₂ | 20 | | | | 3.18 | | | | 4 | | 1.1 | nein |
| 55 | Al₂O₃ | 18.08 | 1.92 | 0.2 | | | | | | | | 2.17 | nein |
| 56 | Al₂O₃ | 20 | | 0.02 | | | 1.02 | 0.1 | | | | 0.83 | nein |
| 57 | Al₂O₃ | 20 | | | | 0.63 | | | | 4 | | 1.31 | nein |
| 58 | TiO₂ | 20 | | 0.2 | | | | | | | | 1.99 | nein |
| 59 | TiO₂ | 20 | | 0.2 | | | | | | | | 1.52 | nein |
| 60 | TiO₂ | 20 | | 0.1 | | | | | | 0.2 | 0.29 | 1.4 | nein |
| 61 | TiO₂ | 20 | | 0.2 | | | | | | | | 0.98 | nein |
| 62 | TiO₂ | 20 | | | | | | 3.27 | 2.96 | 1 | | 1.48 | nein |
| 63 | TiO₂ | 20 | | 0.2 | | | | | | | | 1.16 | nein |

Kat. = catalyst; Träger = carrier; Oxalat = oxalate; nein = no
*: Oxalate was used in the production of the catalyst, but oxalate is not contained in the composition of the calcinated catalyst.

| | | Fe | Co | Ru | Al | La | Mg | Zr | Mn | Cu | Zn | K | |
|---|---|---|---|---|---|---|---|---|---|---|---|---|---|
| Kat. | Träger | in % | in % | in % | in % | in % | in % | in % | in % | in % | in % | in % | Oxalat * |
| 64 | TiO₂ | 20 | | | | | | 3.27 | 2.96 | 1 | | 1.48 | nein |
| 65 | Pural | 20 | | 0.07 | | | 1.27 | | | 1.78 | | 1.74 | nein |
| | MG70 | | | | | | | | | | | | |
| 66 | Pural | 20 | | | | | 4 | | 4 | 4 | | 2.4 | nein |
| | MG70 | | | | | | | | | | | | |
| 67 | Pural | 20 | | 0.07 | | | | | | | 0.3 | 2.1 | nein |
| | MG70 | | | | | | | | | | | | |

Kat. = catalyst; Träger = carrier; Oxalat = oxalate; nein = no
*: Oxalate was used in the production of the catalyst, but oxalate is not contained in the composition of the calcinated catalyst.

### Results of the Catalytic Tests of Catalysts 11-67

The testing of the catalytic properties of catalysts 11-67 in relation to the hydrogenation of carbon dioxide with hydrogen and the analysis of the product gases took place in the same way as the testing and analysis of catalysts 1-10.

The results of the conversion of carbon dioxide with hydrogen in the presence of catalysts 11-67 are listed in the Tables 3 to 7 below. The conversions (X) and yields (Y) were calculated—reagent-based—, and the selectivity (S) was calculated—product based.

TABLE 3
Results of the catalytic tests. Reaction conditions: p = 10 bar, T = 300° C., H₂:CO₂:N₂ = 71:24:5, GHSV = 1320 ml g_{cat}⁻¹ h⁻¹

| | | | | | | | C₃-C₁₅- | | | |
|---|---|---|---|---|---|---|---|---|---|---|
| | | | | | | | Anteil | Olefin- | Olefin- | Olefin- |
| | | | | | | | an den | anteil | anteil | anteil |
| Kat. | X(CO₂) | Y(C1) | Y(C₂-C₄) | Y(C₅-C₁₅) | Y(CO) | S(C₅-C₁₅) | KW | (C₂) | (C₃) | (C₄) |
| 11 | 27% | 1% | 5% | 9% | 7% | 40% | 59% | 44% | 82% | 79% |
| 12 | 27% | 2% | 5% | 8% | 7% | 37% | 55% | 47% | 82% | 79% |
| 13 | 32% | 6% | 11% | 7% | 4% | 25% | 30% | 1% | 15% | 34% |
| 14 | 30% | 5% | 11% | 7% | 5% | 25% | 30% | 8% | 55% | 60% |
| 15 | 30% | 6% | 11% | 7% | 5% | 24% | 29% | 7% | 52% | 59% |
| 16 | 27% | 1% | 5% | 7% | 5% | 35% | 54% | 55% | 82% | 81% |
| 17 | 30% | 5% | 10% | 7% | 5% | 25% | 30% | 4% | 31% | 47% |
| 18 | 30% | 5% | 10% | 7% | 5% | 25% | 30% | 38% | 71% | 76% |
| 19 | 31% | 6% | 11% | 7% | 5% | 24% | 29% | 37% | 79% | 77% |
| 20 | 29% | 6% | 12% | 7% | 4% | 23% | 27% | 2% | 22% | 40% |
| 21 | 30% | 6% | 11% | 6% | 5% | 23% | 27% | 2% | 30% | 47% |
| 22 | 23% | 2% | 4% | 6% | 9% | 30% | 51% | 14% | 65% | 62% |
| 23 | 24% | 2% | 4% | 6% | 9% | 29% | 51% | 10% | 57% | 57% |
| 24 | 27% | 5% | 10% | 6% | 5% | 24% | 30% | 33% | 73% | 75% |
| 25 | 30% | 6% | 12% | 6% | 4% | 22% | 26% | 2% | 13% | 29% |
| 26 | 29% | 6% | 11% | 6% | 4% | 23% | 27% | 6% | 42% | 54% |
| 27 | 25% | 1% | 4% | 6% | 9% | 30% | 55% | 72% | 64% | 83% |
| 28 | 26% | 4% | 8% | 6% | 6% | 24% | 32% | 2% | 24% | 43% |
| 29 | 28% | 5% | 10% | 6% | 5% | 23% | 28% | 26% | 66% | 71% |

Kat. = catalyst;
Anteil an den KW = portion of hydrocarbons;
Olefinanteil = olefin portion

TABLE 4
Results of the catalytic tests. Reaction conditions: p = 10 bar, T = 300° C., H₂:CO₂:N₂ = 71:24:5, GHSV = 1320 ml g_{cat}⁻¹ h⁻¹

| Kat. | X(CO₂) | S(C1) | S(C₂-C₄) | S(C₅-C₁₅) | S(CO) | Y(C₅-C₁₅) − Y(C1) |
|---|---|---|---|---|---|---|
| 30 | 24% | 6% | 19% | 26% | 49% | 4% |
| 31 | 23% | 6% | 17% | 25% | 52% | 3% |
| 32 | 26% | 8% | 24% | 25% | 42% | 3% |
| 33 | 25% | 9% | 23% | 24% | 43% | 3% |
| 34 | 23% | 8% | 20% | 23% | 49% | 3% |
| 35 | 25% | 10% | 26% | 24% | 40% | 3% |
| 36 | 21% | 5% | 15% | 19% | 61% | 3% |
| 37 | 22% | 7% | 19% | 21% | 53% | 3% |
| 38 | 22% | 7% | 20% | 21% | 52% | 2% |
| 39 | 24% | 14% | 33% | 24% | 29% | 2% |
| 40 | 22% | 9% | 21% | 20% | 50% | 2% |
| 41 | 23% | 12% | 26% | 22% | 40% | 2% |
| 42 | 18% | 6% | 15% | 17% | 62% | 2% |

Kat. = catalyst

TABLE 5
Results of the catalytic tests. Reaction conditions: p = 10 bar, T = 350° C., H₂:CO₂ = 3, GHSV = 660 ml g_{cat}⁻¹ h⁻¹

| | Y(C₅-C₁₅) − | | | | | | | | |
|---|---|---|---|---|---|---|---|---|---|
| Kat. | Y(C1) | X(CO₂) | X(H₂) | Y(CO) | Y(CH₄) | Y(C₂-C₄) | Y(C₅-C₁₅) | S(C₂-C₄) | S(C₅-C₁₅) |
| 43 | 5.8% | 42% | 41% | 6.0% | 5.5% | 9.2% | 11.3% | 29% | 35% |
| 44 | 5.8% | 42% | 40% | 6.0% | 5.1% | 9.4% | 10.6% | 30% | 34% |
| 45 | 5.5% | 41% | 41% | 5.4% | 5.6% | 10.5% | 11.2% | 32% | 34% |
| 46 | 4.8% | 39% | 38% | 6.3% | 4.9% | 9.6% | 9.7% | 31% | 32% |
| 47 | 4.9% | 38% | 36% | 7.1% | 4.5% | 8.5% | 9.4% | 29% | 32% |

Kat. = catalyst

TABLE 6
Results of the catalytic tests. Reaction conditions: p = 15 bar, T = 350°
C., H₂: CO₂ = 6, GHSV = 1320 ml g_{cat}⁻¹ h⁻¹

| | Y(C₅-C₁₅) in % | | | |
|---|---|---|---|---|
| | 300° C., | 300° C., | 350° C., | 350° C., |
| Kat. | H₂: CO₂ = 3 | H₂: CO₂ = 6 | H₂: CO₂ = 3 | H₂: CO₂ = 6 |
| 48 | 10.1 | 15.6 | 10.7 | 15.0 |
| 49 | 7.0 | 12.2 | 9.5 | 12.1 |
| 50 | 9.0 | 13.0 | 10.6 | 10.7 |
| 51 | 8.9 | 14.2 | 9.9 | 12.0 |
| 52 | 9.4 | 13.5 | 11.5 | 12.9 |
| 53 | 7.2 | 13.9 | 10.9 | 18.1 |
| 54 | 9.4 | 11.5 | 6.7 | 10.7 |
| 55 | 8.3 | 11.7 | 8.9 | 11.1 |
| 56 | 7.6 | 11.3 | 9.6 | 12.7 |
| 57 | 10.6 | 14.3 | 11.2 | 13.1 |

Kat. = catalyst

TABLE 7
Results of the catalytic tests. Reaction conditions: p = 15 bar, H₂:CO₂ = 6, GHSV = 1320 ml g_{cat}⁻¹ h⁻¹

| | | | | | | | | | Olefin- |
|---|---|---|---|---|---|---|---|---|---|
| | T | | | | | | | S | anteil |
| Kat. | in ° C. | X(CO₂) | X(H₂) | Y(C₅-C₁₅) | Y(C₂-C₄) | Y(CO) | Y(CH₄) | (C₅-C₁₅) | (C₂-C₄) |
| 58 | 350 | 65% | 31% | 18% | 19% | 5% | 11% | 34% | |
| 59 | 350 | 66% | 31% | 18% | 20% | 5% | 12% | 32% | |
| 60 | 350 | 65% | 33% | 17% | 19% | 5% | 11% | 33% | |
| 61 | 300 | 50% | 26% | 17% | 17% | 4% | 6% | 38% | |
| 62 | 300 | 46% | 23% | 16% | 15% | 5% | 5% | 40% | |
| 63 | 350 | 67% | 34% | 16% | 21% | 4% | 13% | 29% | |
| 64 | 350 | 64% | 31% | 16% | 21% | 5% | 12% | 30% | |
| 65 | 350 | 72% | 38% | 22.4% | 24.2% | 3.4% | 17.2% | 33.2% | 83% |
| 66 | 350 | 70% | 36% | 23.1% | 23.0% | 3.8% | 13.8% | 36.1% | 84% |
| 67 | 350 | 73% | 38% | 22.6% | 23.7% | 3.3% | 17.3% | 33.6% | 85% |

Kat. = catalyst;
Olefinanteil = olefin portion

1. A process for producing hydrocarbons from carbon dioxide and hydrogen wherein carbon dioxide is converted with hydrogen in the presence of a catalyst, the catalyst containing iron and at least one alkali metal and furthermore fulfilling at least one of the following features (i) to (iii):
(i) in relation to the overall weight of the catalyst, the catalyst contains iron in an amount of at least 10% by weight, preferably of 15 and 35% by weight, and furthermore copper, and at least one additional representative selected from magnesium, zinc, lanthanum and zirconium;
(ii) the catalyst comprises TiO₂ and/or an aluminum magnesium hydroxycarbonate as a catalyst carrier;
iii) the catalyst also contains ruthenium and/or cobalt.

2. The process according to claim 1, wherein the conversion of the carbon dioxide with hydrogen takes place in the presence of the catalyst at a temperature of 150 to 400° C., preferably 320 to 370° C. and a pressure of 10 to 50 bar, preferably 15 to 30 bar.

3. The process according to claim 1 or 2, wherein the molar ratio of the hydrogen used to carbon dioxide is 4 to 8.

4. The process according to at least one of claims 1 to 3, wherein a product mixture is obtained that contains hydrocarbons, carbon monoxide, non-converted carbon dioxide and non-converted hydrogen, and after separation of the hydrocarbons, non-converted carbon dioxide, non-converted hydrogen and carbon monoxide are delivered back to the reagent mixture.

5. The process according to at least one of claims 1 to 4, wherein the hydrocarbons comprise C₂-C₄ hydrocarbons.

6. The process according to claim 5, wherein the C₂-C₄ hydrocarbons contain C₂-C₄ alkanes and C₂-C₄ alkenes, and after their separation, the C₂-C₄ alkanes are then dehydrogenated to form C₂-C₄ alkenes, in particular ethylene, propylene and butenes.

7. The process according to at least one of claims 1 to 4, wherein the hydrocarbons comprise C₅-C₁₅ hydrocarbons.

8. The process according to at least one of claims 1 to 7, wherein the at least one alkali metal in the catalyst is potassium.

9. The process according to at least one of claims 1 to 8, wherein the catalyst contains:
magnesium;
zinc;
zirconium, manganese and zinc;
copper;
copper and another representative selected from the group consisting of aluminum, lanthanum, zirconium, zinc and magnesium
copper, magnesium and manganese; or
copper, zirconium and manganese.

10. The process according to at least one of claims 1 to 9, wherein the catalyst fulfils feature (i) or (iii) and comprises a catalyst carrier.

11. The process according to claim 10, wherein the catalyst carrier is selected from the group consisting of Al₂O₃, Al₂ O₃ MgO, TiO₂, La₂O₃, ZrO₂, ZrO₂ La₂O₃, ZrO₂ SiO₂, zeolites and aluminum magnesium hydroxycarbonates.

12. The process according to claim 10, wherein the catalyst carrier is selected from the group consisting of TiO₂, Al₂O₃ and aluminum magnesium hydroxycarbonate.

13. A catalyst which, as catalyst components, comprises, in relation to the overall weight of the catalyst, at least 10% by weight, preferably 15-35% by weight iron, as well as potassium, copper and at least one other component, selected from magnesium, zinc, lanthanum and zirconium.

14. The catalyst according to claim 13, which comprises a catalyst carrier to which the catalyst components are applied.

15. The catalyst according to claim 14, the catalyst carrier being selected from the group consisting of TiO₂, Al₂O₃ and/or aluminum magnesium hydroxycarbonate.
